(19) United States
(12) Patent Application Publication
(10) Pub. No.: US 2014/0094675 A1
(43) Pub. Date: Apr. 3, 2014

(54) ARRAYED ELECTRODES IN A WEARABLE DEVICE FOR DETERMINING PHYSIOLOGICAL CHARACTERISTICS

(71) Applicants: Michael Luna, San Jose, CA (US); Scott Fullam, Palo Alto, CA (US)

(72) Inventors: Michael Luna, San Jose, CA (US); Scott Fullam, Palo Alto, CA (US)

(73) Assignee: AliphCom, San Francisco, CA (US)

(21) Appl. No.: 13/831,260

(22) Filed: Mar. 14, 2013

(30) Foreign Application Priority Data
Sep. 29, 2012 (CN) 201220513278.5

(51) Int. Cl. A61B 5/00; A61B 5/04

(52) U.S. Cl. CPC A61B 5/721; A61B 5/04
USPC 600/386

(57) ABSTRACT

Embodiments relate generally to electrical and electronic hardware, computer software, wired and wireless network communications, and wearable computing devices in capturing and deriving physiological characteristic data. More specifically, an array of electrodes and methods are configured to determine physiological characteristics using a wearable device (or carried device) that may be subject to motion. In one embodiment, an array of electrodes is disposed substantially in a wearable housing. At least a portion of the array including electrodes configured to either drive a first signal to a target location or receive a second signal from the target location. The second signal includes data representing one or more physiological characteristics. A sensor selector is configured to identify a subset of the electrodes adjacent to the target location and to select the subset of the electrodes from which to receive a sensor signal that includes data representing one or more physiological characteristics.

### CROSS-RELATED APPLICATIONS

This application claims priority to Chinese Utility Model Patent Application Number 201220513278.5 filed on Sep. 29, 2012, which is incorporated by reference herein for all purposes. This application is also related to U.S. Nonprovisional patent application Ser. No. 13/802,305, filed Mar. 13, 2013, with Attorney Docket No. ALI-267 and U.S. Nonprovisional patent application Ser. No. 13/802,319, filed Mar. 13, 2013, with Attorney Docket No. ALI-268, all of which are incorporated by reference for all purposes.

### FIELD

Embodiments of the invention relate generally to electrical and electronic hardware, computer software, wired and wireless network communications, and wearable computing devices for facilitating health and wellness-related information. More specifically, disclosed are an array of electrodes and methods to determine physiological characteristics using a wearable device (or carried device) that can be subject to motion.

### BACKGROUND

Devices and techniques to gather physiological information, such as a heart rate of a person, while often readily available, are not well-suited to capture such information other than by using conventional data capture devices. Conventional devices typically lack capabilities to capture, analyze, communicate, or use physiological-related data in a contextually-meaningful, comprehensive, and efficient manner, such as during the day-to-day activities of a user, including high impact and strenuous exercising or participation in sports. Further, traditional devices and solutions to obtaining physiological information generally require that the sensors remain firmly affixed to the person, such as being affixed to the skin. In some conventional approaches, a few sensors are placed directly on the skin of a person while the sensors and the person are relatively stationary during the measurement process. While functional, the traditional devices and solutions to collecting physiological information are not well-suited for active participants in sports or over the course of one or more days.

Thus, what is needed is a solution for data capture devices, such as for wearable devices, without the limitations of conventional techniques.

### BRIEF DESCRIPTION OF THE DRAWINGS

Various embodiments or examples (“examples”) of the invention are disclosed in the following detailed description and the accompanying drawings:

FIG. 1A illustrates an exemplary array of electrodes and a physiological information generator disposed in a wearable data-capable band, according to some embodiments;

FIGS. 1B to 1D illustrate examples of electrode arrays, according to some embodiments;

FIG. 2 is a functional diagram depicting a physiological information generator implemented in a wearable device, according to some embodiments;

FIGS. 3A to 3C are cross-sectional views depicting arrays of electrodes including subsets of electrodes adjacent an arm of a wearer, according to some embodiments;

FIG. 4 depicts a portion of an array of electrodes disposed within a housing material of a wearable device, according to some embodiments;

FIG. 5 depicts an example of a physiological information generator, according to some embodiments;

FIG. 6 is an example flow diagram for selecting a sensor, according to some embodiments;

FIG. 7 is an example flow diagram for determining physiological characteristics using a wearable device with arrayed electrodes, according to some embodiments; and

FIG. 8 illustrates an exemplary computing platform disposed in a wearable device in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

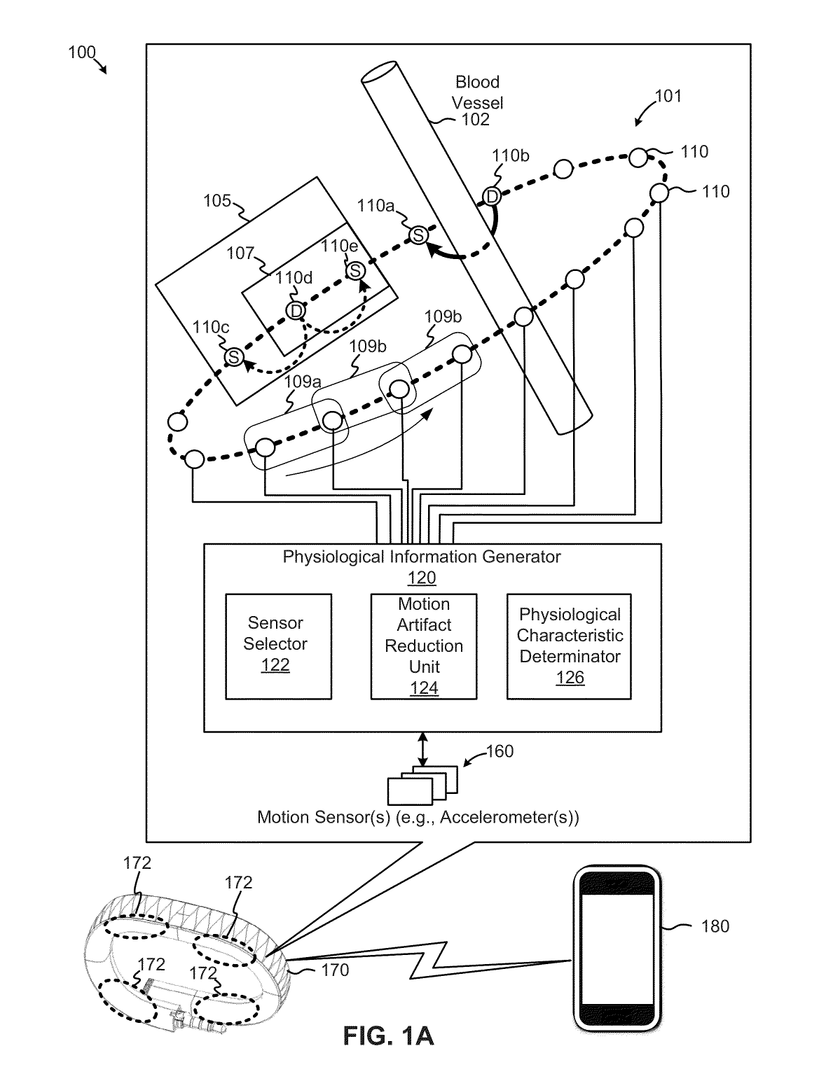

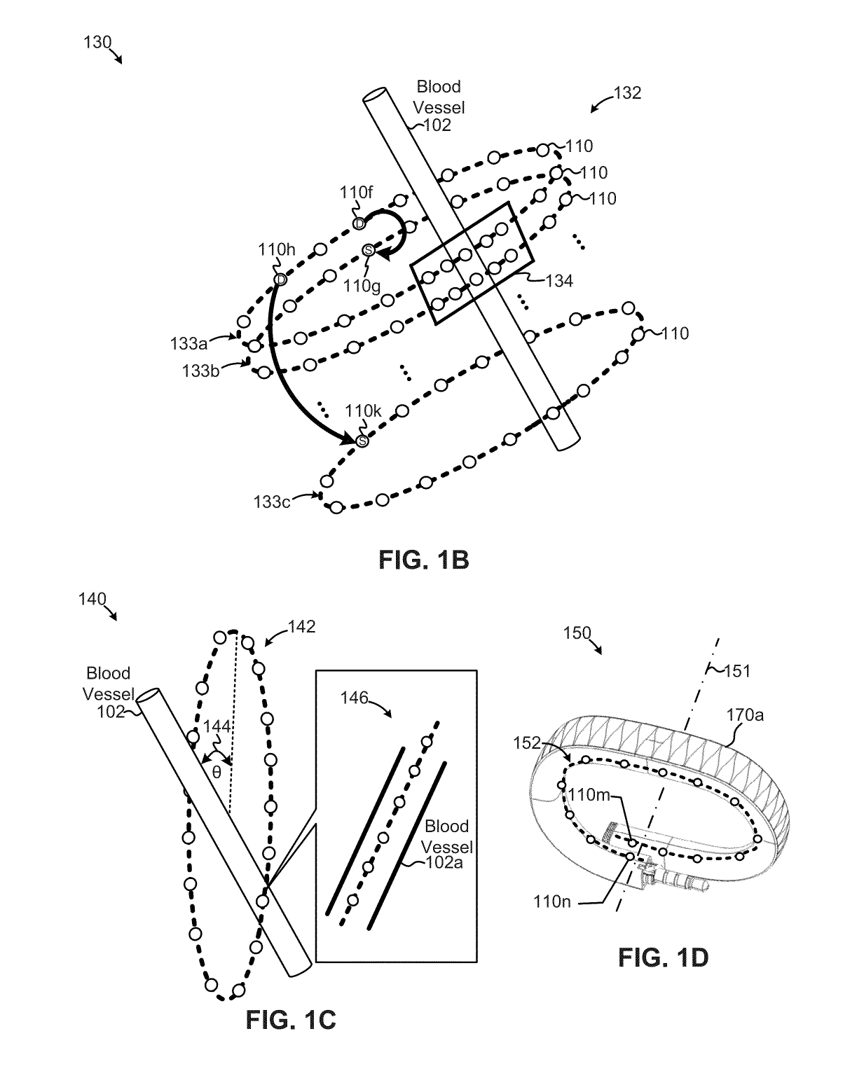

FIG. 1A illustrates an exemplary array of electrodes and a physiological information generator disposed in a wearable data-capable band, according to some embodiments. Diagram 100 depicts an array 100 of electrodes 110 coupled to a physiological information generator 120 that is configured to generate data representing one or more physiological characteristics associated with a user that is wearing or carrying array 101. Also shown are motion sensors 160, which, for example, can include accelerometers. Motion sensors 160 are not limited to accelerometers. Examples of motion sensors 160 can also include gyroscopic sensors, optical motion sensors (e.g., laser or LED motion detectors, such as used in optical mice), magnet-based motion sensors (e.g., detecting magnetic fields, or changes thereof, to detect motion), electromagnetic-based sensors, etc., as well as any sensor configured to detect or determine motion, such as motion sensors based on physiological characteristics (e.g., using electromyography (“EMG”) to determine existence and/or amounts of motion based on electrical signals generated by muscle cells), and the like. Electrodes 110 can include any suitable structure for transferring signals and picking up signals, regardless of whether the signals are electrical, magnetic, optical, pressure-based, physical, acoustic, etc., according to various embodiments. According to some embodiments, electrodes 110 of array 101 are configured to couple capacitively to a target location. In some embodiments, array 101 and physiological information generator 120 are disposed in a wearable device, such as a wearable data-capable band 170, which may include a housing that encapsulates, or substantially encapsulates, array 101 of electrodes 110. In operations, physiological information generator 120 can determine the bioelectric impedance (“bioimpedance”) of one or more types of tissues of a wearer to identify, measure, and monitor physiological characteristics. For example, a drive signal having a known amplitude and frequency can be applied to a user, from which a sink signal is received as bioimpedance signal. The bioimpedance signal is a measured signal that includes real and complex components. Examples of real components include extra-cellular and intra-cellular spaces of tissue, among other things, and examples of complex components include cellular membrane capacitance, among other things. Further, the measured bioimpedance signal can include real and/or complex components associated with arterial structures (e.g., arterial cells, etc.) and the presence (or absence) of blood pulsing through an arterial structure. In some examples, a heart rate signal, or other physiological signals, can be determined (i.e., recovered) from the measured bioimpedance signal by, for example, comparing the measured bioimpedance signal against the waveform of the drive signal to determine a phase delay (or shift) of the measured complex components.

Physiological information generator 120 is shown to include a sensor selector 122, a motion artifact reduction unit 124, and a physiological characteristic determinator 126. Sensor selector 122 is configured to select a subset of electrodes, and is further configured to use the selected subset of electrodes to acquire physiological characteristics, according to some embodiments. Examples of a subset of electrodes include subset 107, which is composed of electrodes 110d and 110e, and subset 105, which is composed of electrodes 110c, 110d and 110e. More or fewer electrodes can be used. Sensor selector 122 is configured to determine which one or more subsets of electrodes 110 (out of a number of subsets of electrodes 110) are adjacent to a target location. As used herein, the term “target location” can, for example, refer to a region in space from which a physiological characteristic can be determined. A target region can be adjacent to a source of the physiological characteristic, such as blood vessel 102, with which an impedance signal can be captured and analyzed to identify one or more physiological characteristics. The target region can reside in two-dimensional space, such as an area on the skin of a user adjacent to the source of the physiological characteristic, or in three-dimensional space, such as a volume that includes the source of the physiological characteristic. Sensor selector 122 operates to either drive a first signal via a selected subset to a target location, or receive a second signal from the target location, or both. The second signal includes data representing one or more physiological characteristics. For example, sensor selector 122 can configure electrode (“D”) 110b to operate as a drive electrode that drives a signal (e.g., an AC signal) into the target location, such as into the skin of a user, and can configure electrode (“S”) 110a to operate as a sink electrode (i.e., a receiver electrode) to receive a second signal from the target location, such as from the skin of the user. In this configuration, sensor selector 112 can drive a current signal via electrode (“D”) 110b into a target location to cause a current to pass through the target location to another electrode (“S”) 110a. In various examples, the target location can be adjacent to or can include blood vessel 102. Examples of blood vessel 102 include a radial artery, an ulnar artery, or any other blood vessel. Array 101 is not limited to being disposed adjacent blood vessel 102 in an arm, but can be disposed on any portion of a user's person (e.g., on an ankle, ear lobe, around a finger or on a fingertip, etc.). Note that each electrode 110 can be configured as either a driver or a sink electrode. Thus, electrode 110b is not limited to being a driver electrode and can be configured as a sink electrode in some implementations. As used herein, the term “sensor” can refer, for example, to a combination of one or more driver electrodes and one or more sink electrodes for determining one or more bioimpedance-related values and/or signals, according to some embodiments.

In some embodiments, sensor selector 122 can be configured to determine (periodically or aperiodically) whether the subset of electrodes 110a and 110b are optimal electrodes 110 for acquiring a sufficient representation of the one or more physiological characteristics from the second signal. To illustrate, consider that electrodes 110a and 110b may be displaced from the target location when, for instance, wearable device 170 is subject to a displacement in a plane substantially perpendicular to blood vessel 102. The displacement of electrodes 110a and 110b may increase the impedance (and/or reactance) of a current path between the electrodes 110a and 110b, or otherwise move those electrodes away from the target location far enough to degrade or attenuate the second signals retrieved therefrom. While electrodes 110a and 110b may be displaced from the target location, other electrodes are displaced to a position previously occupied by electrodes 110a and 110b (i.e., adjacent to the target location). For example, electrodes 110c and 110d may be displaced to a position adjacent to blood vessel 102. In this case, sensor selector 122 operates to determine an optimal subset of electrodes 110, such as electrodes 110c and 110d, to acquire the one or more physiological characteristics. Therefore, regardless of the displacement of wearable device 170 about blood vessel 102, sensor selector 122 can repeatedly determine an optimal subset of electrodes for extracting physiological characteristic information from adjacent a blood vessel. For example, sensor selector 122 can repeatedly test subsets in sequence (or in any other matter) to determine which one is disposed adjacent to a target location. For example, sensor selector 122 can select at least one of subset 109a, subset 109b, subset 109c, and other like subsets, as the subset from which to acquire physiological data.

According to some embodiments, array 101 of electrodes can be configured to acquire one or more physiological characteristics from multiple sources, such as multiple blood vessels. To illustrate, consider that, for example, blood vessel 102 is an ulnar artery adjacent electrodes 110a and 110b and a radial artery (not shown) is adjacent electrodes 110c and 110d. With multiple sources of physiological characteristic information being available, there are thus multiple target locations. Therefore, sensor selector 122 can select multiple subsets of electrodes 110, each of which is adjacent to one of a multiple number of target locations. Physiological information generator 120 then can use signal data from each of the multiple sources to confirm accuracy of data acquired, or to use one subset of electrodes (e.g., associated with a radial artery) when one or more other subsets of electrodes (e.g., associated with an ulnar artery) are unavailable.

Note that the second signal received into electrode 110a can be composed of a physiological-related signal component and a motion-related signal component, if array 101 is subject to motion. The motion-related component includes motion artifacts or noise induced into an electrode 110a. Motion artifact reduction unit 124 is configured to receive motion-related signals generated at one or more motion sensors 160, and is further configured to receive at least the motion-related signal component of the second signal. Motion artifact reduction unit 124 operates to eliminate the magnitude of the motion-related signal component, or to reduce the magnitude of the motion-related signal component relative to the magnitude of the physiological-related signal component, thereby yielding as an output the physiological-related signal component (or an approximation thereto). Thus, motion artifact reduction unit 124 can reduce the magnitude of the motion-related signal component (i.e., the motion artifact) by an amount associated with the motion-related signal generated by one or more accelerometers to yield the physiological-related signal component.

Physiological characteristic determinator 126 is configured to receive the physiological-related signal component of the second signal and is further configured to process (e.g., digitally) the signal data including one or more physiological characteristics to derive physiological signals, such as either a heart rate (“HR”) signal or a respiration signal, or both. For example, physiological characteristic determinator 126 is configured to amplify and/or filter the physiological-related component signals (e.g., at different frequency ranges) to extract certain physiological signals. According to various embodiments, a heart rate signal can include (or can be based on) a pulse wave. A pulse wave includes systolic components based on an initial pulse wave portion generated by a contracting heart, and diastolic components based on a reflected wave portion generated by the reflection of the initial pulse wave portion from other limbs. In some examples, an HR signal can include or otherwise relate to an electrocardiogram (“ECG”) signal. Physiological characteristic determinator 126 is further configured to calculate other physiological characteristics based on the acquired one or more physiological characteristics. Optionally, physiological characteristic determinator 126 can use other information to calculate or derive physiological characteristics. Examples of the other information include motion-related data, including the type of activity in which the user is engaged, such as running or sleep, location-related data, environmental-related data, such as temperature, atmospheric pressure, noise levels, etc., and any other type of sensor data, including stress-related levels and activity levels of the wearer.

In some cases, a motion sensor 160 can be disposed adjacent to the target location (not shown) to determine a physiological characteristic via motion data indicative of movement of blood vessel 102 through which blood pulses to identify a heart rate-related physiological characteristic. Motion data, therefore, can be used to supplement impedance determinations of to obtain the physiological characteristic. Further, one or more motion sensors 160 can also be used to determine the orientation of wearable device 170, and relative movement of the same to determine or predict a target location. By predicting a target location, sensor selector 122 can use the predicted target location to begin the selection of optimal subsets of electrodes 110 in a manner that reduces the time to identify a target location.

In view of the foregoing, the functions and/or structures of array 101 of electrodes and physiological information generator 120, as well as their components, can facilitate the acquisition and derivation of physiological characteristics in situ—during which a user is engaged in physical activity that imparts motion on a wearable device, thereby exposing the array of electrodes to motion-related artifacts. Physiological information generator 120 is configured to dampen or otherwise negate the motion-related artifacts from the signals received from the target location, thereby facilitating the provision of heart-related activity and respiration activity to the wearer of wearable device 170 in real-time (or near real-time). As such, the wearer of wearable device 170 need not be stationary or otherwise interrupt an activity in which the wearer is engaged to acquire health-related information. Also, array 101 of electrodes 110 and physiological information generator 120 are configured to accommodate displacement or movement of wearable device 170 about, or relative to, one or more target locations. For example, if the wearer intentionally rotates wearable device 170 about, for example, the wrist of the user, then initial subsets of electrodes 110 adjacent to the target locations (i.e., before the rotation) are moved further away from the target location. As another example, the motion of the wearer (e.g., impact forces experienced during running) may cause wearable device 170 to travel about the wrist. As such, physiological information generator 120 is configured to determine repeatedly whether to select other subsets of electrodes 110 as optimal subsets of electrodes 110 for acquiring physiological characteristics. For example, physiological information generator 120 can be configured to cycle through multiple combinations of driver electrodes and sink electrodes (e.g., subsets 109a, 109b, 109c, etc.) to determine optimal subsets of electrodes. In some embodiments, electrodes 110 in array 101 facilitate physiological data capture irrespective of the gender of the wearer. For example, electrodes 110 can be disposed in array 101 to accommodate data collection of a male or female were irrespective of gender-specific physiological dimensions. In at least one embodiment, data representing the gender of the wearer can be accessible to assist physiological information generator 120 in selecting the optimal subsets of electrodes 110. While electrodes 110 are depicted as being equally-spaced, array 101 is not so limited. In some embodiments, electrodes 110 can be clustered more densely along portions of array 101 at which blood vessels 102 are more likely to be adjacent. For example, electrodes 110 may be clustered more densely at approximate portions 172 of wearable device 170, whereby approximate portions 172 are more likely to be adjacent a radial or ulnar artery than other portions. While wearable device 170 is shown to have an elliptical-like shape, it is not limited to such a shape and can have any shape.

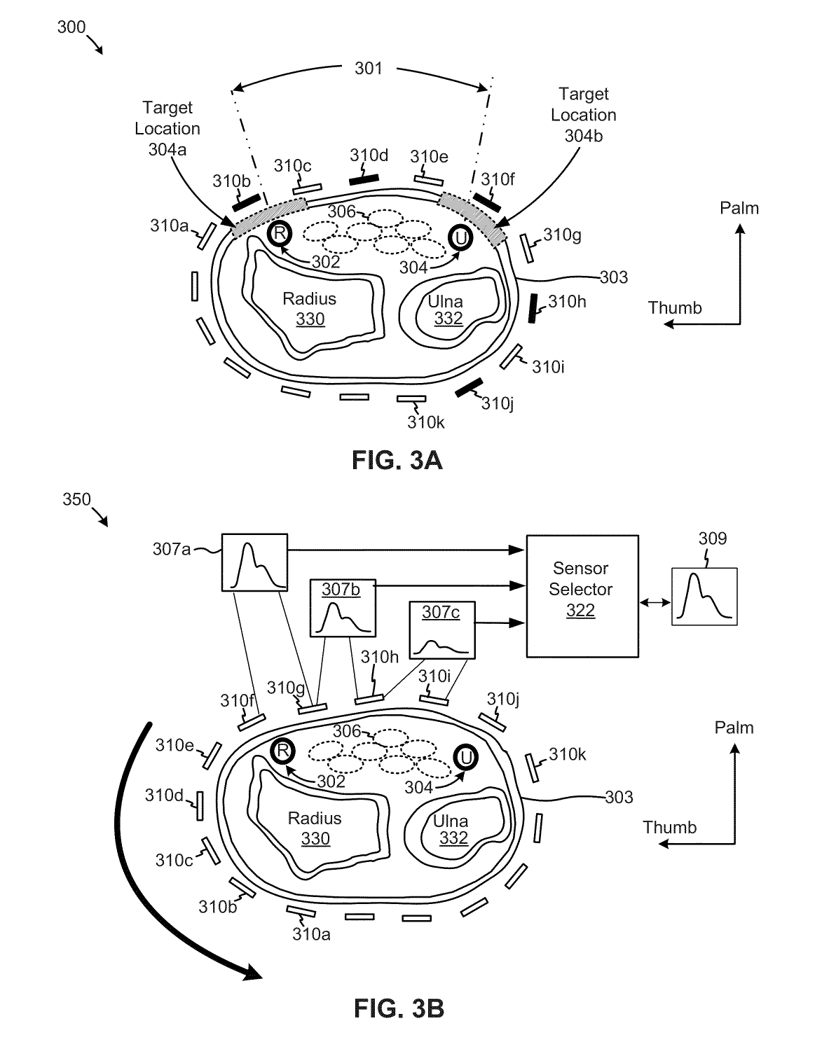

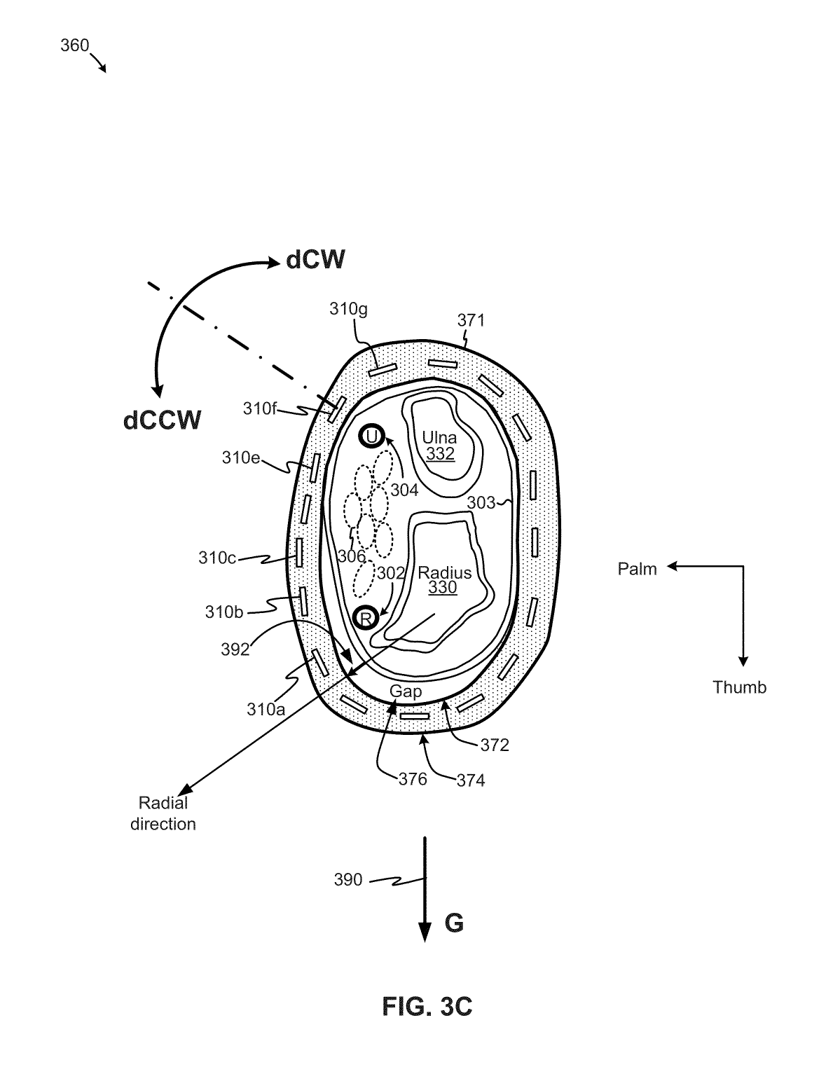

In some instances, a wearable device 170 can select multiple subsets of electrodes to enable data capture using a second subset adjacent to a second target location when a first subset adjacent a first target location is unavailable to capture data. For example, a portion of wearable device 170 including the first subset of electrodes 110 (initially adjacent to a first target location) may be displaced to a position farther away in a radial direction away from a blood vessel, such as depicted by a radial distance 392 of FIG. 3C from the skin of the wearer. That is, subset of electrodes 310a and 310b are displaced radially be distance 392. Further to FIG. 3C, the second subset of electrodes 310f and 310g adjacent to the second target location can be closer in a radial direction toward another blood vessel, and, thus, the second subset of electrodes can acquire physiological characteristics when the first subset of electrodes cannot. Referring back to FIG. 1A, array 101 of electrodes 110 facilitates a wearable device 170 that need not be affixed firmly to the wearer. That is, wearable device 170 can be attached to a portion of the wearer in a manner in which wearable device 170 can be displaced relative to a reference point affixed to the wearer and continue to acquire and generate information regarding physiological characteristics. In some examples, wearable device 170 can be described as being “loosely fitting” on or “floating” about a portion of the wearer, such as a wrist, whereby array 101 has sufficient sensors points from which to pick up physiological signals.

In addition, accelerometers 160 can be used to replace the implementation of subsets of electrodes to detect motion associated with pulsing blood flow, which, in turn, can be indicative of whether oxygen-rich blood is present or not present. Or, accelerometers 160 can be used to supplement the data generated by acquired one or more bioimpedance signals acquired by array 101. Accelerometers 160 can also be used to determine the orientation of wearable device 170 and relative movement of the same to determine or predict a target location. Sensor selector 122 can use the predicted target location to begin the selection of the optimal subsets of electrodes 110, which likely decreases the time to identify a target location. Electrodes 110 of array 101 can be disposed within a material constituting, for example, a housing, according to some embodiments. Therefore, electrodes 110 can be protected from the environment and, thus, need not be subject to corrosive elements. In some examples, one or more electrodes 110 can have at least a portion of a surface exposed. As electrodes 110 of array 101 are configured to couple capacitively to a target location, electrodes 110 thereby facilitate high impedance signal coupling so that the first and second signals can pass through fabric and hair. As such, electrodes 110 need not be limited to direct contact with the skin of a wearer. Further, array 101 of electrodes 110 need not circumscribe a limb or source of physiological characteristics. An array 101 can be linear in nature, or can configurable to include linear and curvilinear portions.

In some embodiments, wearable device 170 can be in communication (e.g., wired or wirelessly) with a mobile device 180, such as a mobile phone or computing device. In some cases, mobile device 180, or any networked computing device (not shown) in communication with wearable device 170 or mobile device 180, can provide at least some of the structures and/or functions of any of the features described herein. As depicted in FIG. 1A and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1A (or any subsequent figure) can represent one or more algorithms. Or, at least one of the elements can represent a portion of logic including a portion of hardware configured to provide constituent structures and/or functionalities.

For example, physiological information generator 120 and any of its one or more components, such as sensor selector 122, motion artifact reduction unit 124, and physiological characteristic determinator 126, can be implemented in one or more computing devices (i.e., any mobile computing device, such as a wearable device or mobile phone, whether worn or carried) that include one or more processors configured to execute one or more algorithms in memory. Thus, at least some of the elements in FIG. 1A (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, physiological information generator 120, including one or more components, such as sensor selector 122, motion artifact reduction unit 124, and physiological characteristic determinator 126, can be implemented in one or more computing devices that include one or more circuits. Thus, at least one of the elements in FIG. 1A (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

FIGS. 1B to 1D illustrate examples of electrode arrays, according to some embodiments. Diagram 130 of FIG. 1B depicts an array 132 that includes sub-arrays 133a, 133b, and 133c of electrodes 110 that are configured to generate data that represent one or more characteristics associated with a user associated with array 132. In various embodiments, drive electrodes and sink electrodes can be disposed in the same sub-array or in different sub-arrays. Note that arrangements of sub-arrays 133a, 133b, and 133c can denote physical or spatial orientations and need not imply electrical, magnetic, or cooperative relationships among electrodes 110 within each sub-array. For example, drive electrode (“D”) 110f can be configured in sub-array 133a as a drive electrode to drive a signal to sink electrode (“S”) 110g in sub-array 133b. As another example, drive electrode (“D”) 110h can be configured in sub-array 133a to drive a signal to sink electrode (“S”) 110k in sub-array 133c. In some embodiments, distances between electrodes 110 in sub-arrays can vary at different regions, including a region in which the placement of electrode group 134 near blood vessel 102 is more probable relative to the placement of other electrodes near blood vessel 102. Electrode group 134 can include a higher density of electrodes 110 than other portions of array 132 as group 134 can be expected to be disposed adjacent blood vessel 102 more likely than other groups of electrodes 110. For example, an elliptical-shaped array (not shown) can be disposed in device 170 of FIG. 1A. Therefore, group 134 of electrodes is disposed at a region 172 of FIG. 1A, which is likely adjacent either a radial artery or an ulna artery. While three sub-arrays are shown, more or fewer are possible.

Referring to FIG. 1C, diagram 140 depicts an array 142 oriented at any angle (“θ”) 144 to an axial line coincident with or parallel to blood vessel 102. Therefore, an array 142 of electrodes need not be oriented orthogonally in each implementation; rather array 142 can be oriented at angles between 0 and 90 degrees, inclusive thereof. In a specific embodiment, an array 146 can be disposed parallel (or substantially parallel) to blood vessel 102a (or a portion thereof).

FIG. 1D is a diagram 150 depicting a wearable device 170a including a helically-shaped array 152 of electrodes disposed therein, whereby electrodes 110m and 110n can be configured as a pair of drive and sink electrodes. As shown, electrodes 110m and 110n substantially align in a direction parallel to an axis 151, which can represent a general direction of blood flow through a blood vessel.

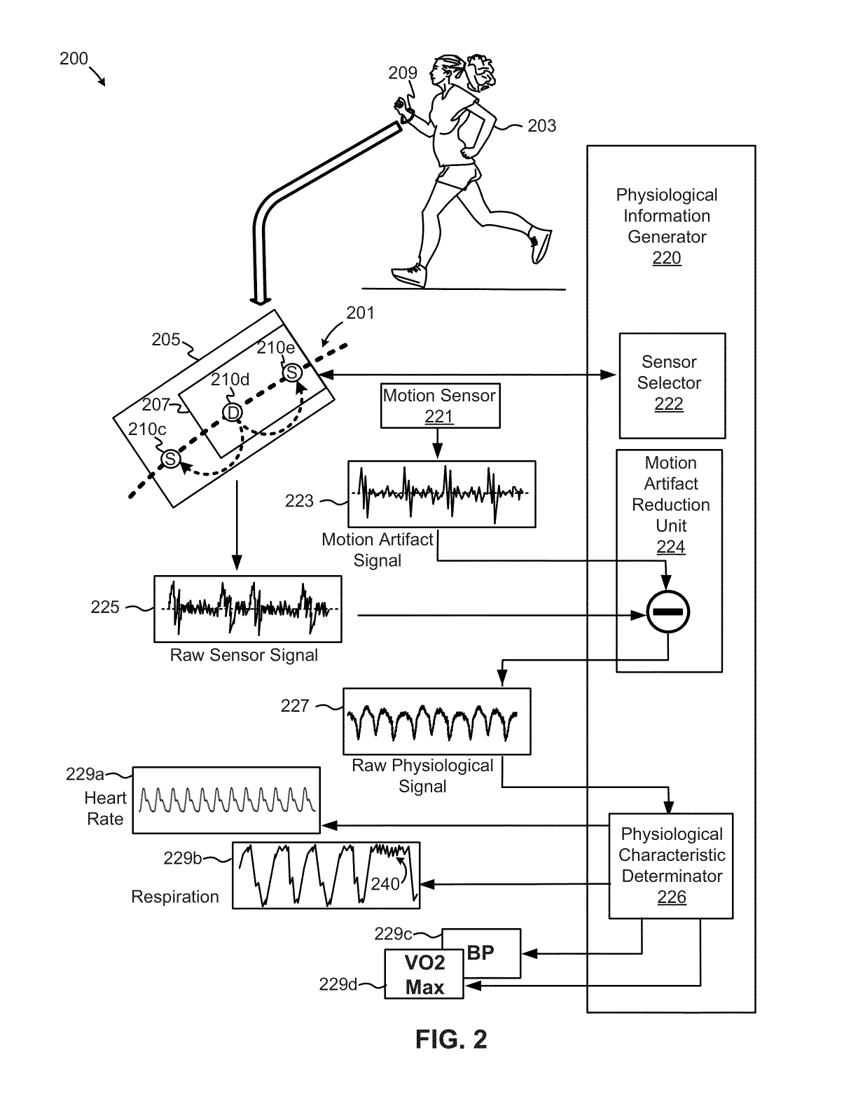

FIG. 2 is a functional diagram depicting a physiological information generator implemented in a wearable device, according to some embodiments. Functional diagram 200 depicts a user 203 wearing a wearable device 209, which includes a physiological information generator 220 configured to generate signals including data representing physiological characteristics. As shown, sensor selector 222 is configured to select a subset 205 of electrodes or a subset 207 of electrodes. Subset 205 of electrodes includes electrodes 210c, 210d, and 210e, and subset 207 of electrodes includes electrodes 210d and 210e. For purposes of illustration, consider that sensor selector 222 selects electrodes 210d and 210c as a subset of electrodes with which to capture physiological characteristics adjacent a target location. Sensor selector 222 applies an AC signal, as a first signal, into electrodes 210d to generate a sensor signal (“raw sensor signal”) 225, as a second signal, from electrode 210c. Sensor signal 222 includes a motion-related signal component and a physiological-related signal component. A motion sensor 221 is configured to capture generate a motion artifact signal 223 based on motion data representing motion experienced by wearable device 209 (or at least the electrodes). A motion artifact reduction unit 224 is configured to receive sensor signal 225 and motion artifact signal 223. Motion artifact reduction unit 224 operates to subtract motion artifact signal 223 from sensor signal 225 to yield the physiological-related signal component (or an approximation thereof) as a raw physiological signal 227. In some examples, raw physiological signal 227 represents an unamplified, unfiltered signal including data representative of one or more physiological characteristics. A physiological characteristic determinator 226 is configured to receive raw physiological signal 227 to amplify and/or filter different physiological signal components from raw physiological signal 227. For example, raw physiological signal 227 may include a respiration signal modulated on (or in association with) a heart rate (“HR”) signal. Regardless, physiological characteristic determinator 226 is configured to perform digital signal processing to generate a heart rate (“HR”) signal 229a and/or a respiration signal 229b. Portion 240 of respiration signal 229b represents an impedance signal due to cardiac activity, at least in some instances. Further, physiological characteristic determinator 226 is configured to use either HR signal 229a or a respiration signal 229b, or both, to derive other physiological characteristics, such as blood pressure data (“BP”) 229c, a maximal oxygen consumption (“VO2 max”) 229d, or any other physiological characteristic.

Physiological characteristic determinator 226 can derive other physiological characteristics using other data generated or accessible by wearable device 209, such as the type of activity the wear is engaged, environmental factors, such as temperature, location, etc., whether the wearer is subject to any chronic illnesses or conditions, and any other health or wellness-related information. For example, if the wearer is diabetic or has Parkinson's disease, motion sensor 221 can be used to detect tremors related to the wearer's ailment. With the detection of small, but rapid movements of a wearable device that coincide with a change in heart rate (e.g., a change in an HR signal) and/or breathing, physiological information generator 220 may generate data (e.g., an alarm) indicating that the wearer is experiencing tremors. For a diabetic, the wearer may experience shakiness because the blood-sugar level is extremely low (e.g., it drops below a range of 38 to 42 mg/dl). Below these levels, the brain may become unable to control the body. Moreover, if the arms of a wearer shakes with sufficient motion to displace a subset of electrodes from being adjacent a target location, the array of electrodes, as described herein, facilitates continued monitoring of a heart rate by repeatedly selecting subsets of electrodes that are positioned optimally (e.g., adjacent a target location) for receiving robust and accurate physiological-related signals.

FIGS. 3A to 3C are cross-sectional views depicting arrays of electrodes including subsets of electrodes adjacent an arm portion of a wearer, according to some embodiments. Diagram 300 of FIG. 3A depicts an array of electrodes arranged about, for example, a wrist of a wearer. In this cross-sectional view, an array of electrodes includes electrodes 310a, 310b, 310c, 310d, 310e, 310f, 310g, 310h, 310i, 310j, and 310k, among others, arranged about wrist 303 (or the forearm). The cross-sectional view of wrist 303 also depicts a radius bone 330, an ulna bone 332, flexor muscles/ligaments 306, a radial artery (“R”) 302, and an ulna artery (“UY”) 304. Radial artery 302 is at a distance 301 (regardless of whether linear or angular) from ulna artery 304. Distance 301 may be different, on average, for different genders, based on male and female anatomical structures. Notably, the array of electrodes can obviate specific placement of electrodes due to different anatomical structures based on gender, preference of the wearer, issues associated with contact (e.g., contact alignment), or any other issue that affects placement of electrode that otherwise may not be optimal. To effect appropriate electrode selection, a sensor selector, as described herein, can use gender-related information (e.g., whether the wearer is male or female) to predict positions of subsets of electrodes such that they are adjacent (or substantially adjacent) to one or more target locations 304a and 304b. Target locations 304a and 304b represent optimal areas (or volumes) at which to measure, monitor and capture data related to bioimpedances. In particular, target location 304a represents an optimal area adjacent radial artery 302 to pick up bioimpedance signals, whereas target location 304b represents another optimal area adjacent ulna artery 304 to pick up other bioimpedance signals.

To illustrate the resiliency of a wearable device to maintain an ability to monitor physiological characteristics over one or more displacements of the wearable device (e.g., around or along wrist 303), consider that a sensor selector configures initially electrodes 310b, 310d, 310f, 310h, and 310j as driver electrodes and electrodes 310a, 310c, 310e 310g, 310i, and 310k as sink electrodes. Further consider that the sensor selector identifies a first subset of electrodes that includes electrodes 310b and 310c as a first optimal subset, and also identifies a second subset of electrodes that include electrodes 310f and 310g as a second optimal subset. Note that electrodes 310b and 310c are adjacent target location 304a and electrodes 310f and 3100g are adjacent to target location 304b. These subsets are used to periodically (or aperiodically) monitor the signals from electrodes 310c and 310g, until the first and second subsets are no longer optimal (e.g., when movement of the wearable device displaces the subsets relative to the target locations). Note that the functionality of driver and sink electrodes for electrodes 310b, 310c, 310f, and 310g can be reversed (e.g., electrodes 310a and 310g can be configured as drive electrodes).

FIG. 3B depicts an array of FIG. 3A being displaced from an initial position, according to some examples. In particular, diagram 350 depicts that electrodes 310f and 3100g are displaced to a location adjacent radial artery 302 and electrodes 310j and 310k are displaced to a location adjacent ulna artery 304. According to some embodiments, a sensor selector 322 is configured to test subsets of electrodes to determine at least one subset, such as electrodes 310f and 310, being located adjacent to a target location (next to radial artery 302). To identify electrodes 310f and 310g as an optimal subset, sensor selector 322 is configured to apply drive signals to the drive electrodes to generate a number of data samples, such as data samples 307a, 307b, and 307c. In this example, each data sample represents a portion of a physiological characteristic, such as a portion of an HR signal. Sensor selector 322 operates to compare the data samples against a profile 309 to determine which of data samples 307a, 307b, and 307c best fits or is comparable to a predefined set of data represented by profile data 309. Profile data 309, in this example, represents an expected HR portion or thresholds indicating a best match. Also, profile data 309 can represent the most robust and accurate HR portion measured during the sensor selection mode relative to all other data samples (e.g., data sample 307a is stored as profile data 309 until, and if, another data sample provides a more robust and/or accurate data sample). As shown, data sample 307a substantially matches profile data 309, whereas data samples 307b and 307c are increasingly attenuated as distances increase away from radial artery 302. Therefore, sensor selector 322 identifies electrodes 310f and 310g as an optimal subset and can use this subset in data capture mode to monitor (e.g., continuously) the physiological characteristics of the wearer. Note that the nature of data samples 307a, 307b, and 307c as portions of an HR signal is for purposes of explanation and is not intended to be limiting. Data samples 307a, 307b, and 307c need not be portions of a waveform or signal, and need not be limited to an HR signal. Rather, data samples 307a, 307b, and 307c can relate to a respiration signal, a raw sensor signal, a raw physiological signal, or any other signal. Data samples 307a, 307b, and 307c can represent a measured signal attribute, such as magnitude or amplitude, against which profile data 309 is matched. In some cases, an optimal subset of electrodes can be associated with a least amount of impedance and/or reactance (e.g., over a period of time) when applying a first signal (e.g., a drive signal) to a target location.

FIG. 3C depicts an array of electrodes of FIG. 3A oriented differently due to a change in orientation of a wrist of a wearer, according to some examples. In this example, the array of electrodes is shown to be disposed in a wearable device 371, which has an outer surface 374 and an inner surface 372. In some embodiments, wearable device 371 can be configured to “loosely fit” around the wrist, thereby enabling rotation about the wrist. In some cases, a portion of wearable devices 371 (and corresponding electrodes 310a and 310b) are subject to gravity (“G”) 390, which pulls the portion away from wrist 303, thereby forming a gap 376. Gap 376, in turn, causes inner surface 372 and electrodes 310a and 310b to be displaced radially by a radial distance 392 (i.e., in a radial direction away from wrist 303). Gap 376, in some cases, can be an air gap. Radial distance 392, at least in some cases, may impact electrodes 310a and 310b and the ability to receive signals adjacent to radial artery 302. Regardless, electrodes 310f and 3100g are positioned in another portion of wearable device 371 and can be used to receive signals adjacent to ulna artery 304 in cooperation with, or instead of, electrodes 310a and 310b. Therefore, electrodes 310f and 310g (or any other subset of electrodes) can provide redundant data capturing capabilities should other subsets be unavailable.

Next, consider that sensor selector 322 of FIG. 3B is configured to determine a position of electrodes 310f and 310g (e.g., on the wearable device 371) relative to a direction of gravity 390. A motion sensor (not shown) can determine relative movements of the position of electrodes 3100f and 310g over any number of movements in either a clockwise direction (“dCW”) or a counterclockwise direction (“dCCW”). As wearable device 371 need not be affixed firmly to wrist 303, at least in some examples, the position of electrodes 310f and 310g may “slip” relative to the position of ulna artery 304. In one embodiment, sensor selector 322 can be configured to determine whether another subset of electrodes are optimal, if electrodes 3100f and 310g are displaced farther away than a more suitable subset. In sensor selecting mode, sensor selector 322 is configured to select another subset, if necessary, by beginning the capture of data samples at electrodes 310f and 310g and progressing to other nearby subsets to either confirm the initial selection of electrodes 310f and 310g or to select another subset. In this manner, the identification of the optimal subset may be determined in less time than if the selection process is performed otherwise (e.g., beginning at a specific subset regardless of the position of the last known target location).

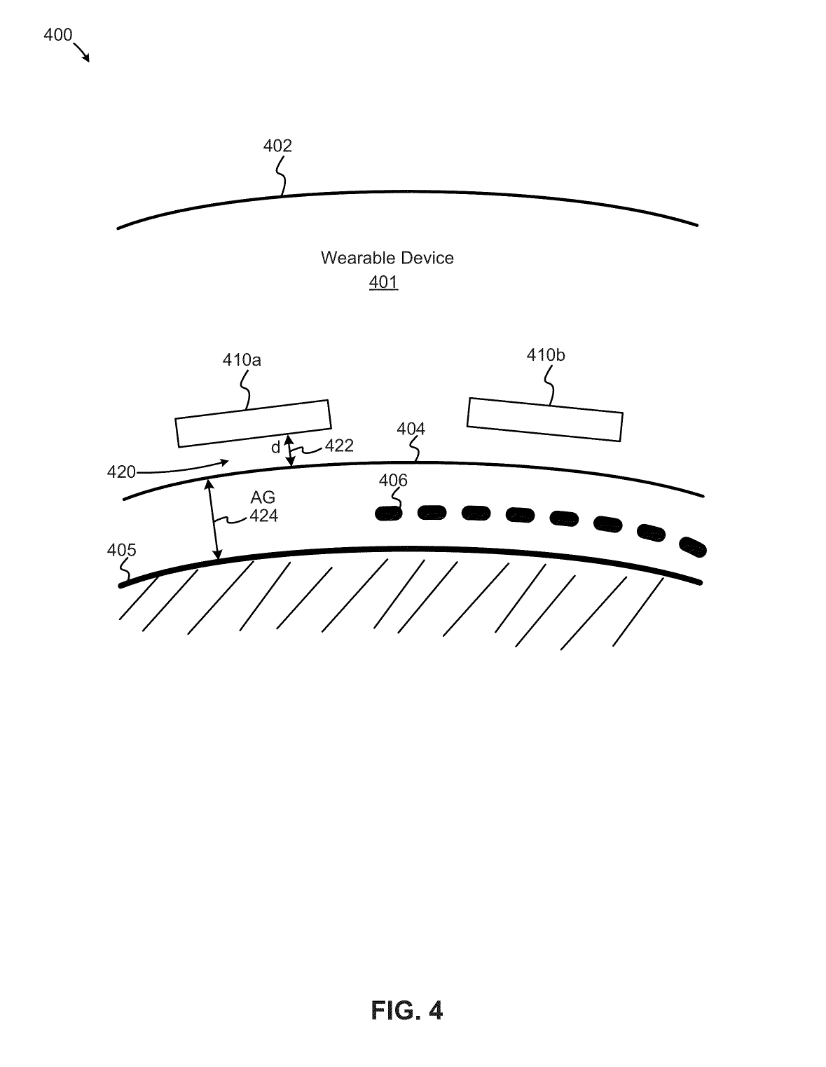

FIG. 4 depicts a portion of an array of electrodes disposed within a housing material of a wearable device, according to some embodiments. Diagram 400 depicts electrodes 410a and 410b disposed in a wearable device 401, which has an outer surface 402 and an inner surface 404. In some embodiments, wearable device 401 includes a material in which electrodes 410a and 410b can be encapsulated in a material to reduce or eliminate exposure to corrosive elements in the environment external to wearable device 401. Therefore, material 420 is disposed between the surfaces of electrodes 410a and 410b and inner surface 404. Driver electrodes are capacitively coupled to skin 405 to transmit high impedance signals, such as a current signal, over distance (“d”) 422 through the material, and, optionally, through fabric 406 or hair into skin 405 of the wearer. Also, the current signal can be driven through an air gap (“AG”) 424 between inner surface 404 and skin 405. Note that in some implementations, electrodes 410a and 410b can be exposed (or partially exposed) out through inner surface 404. In some embodiments, electrodes 410a and 410b can be coupled via conductive materials, such as conductive polymers or the like, to the external environment of wearable device 401.

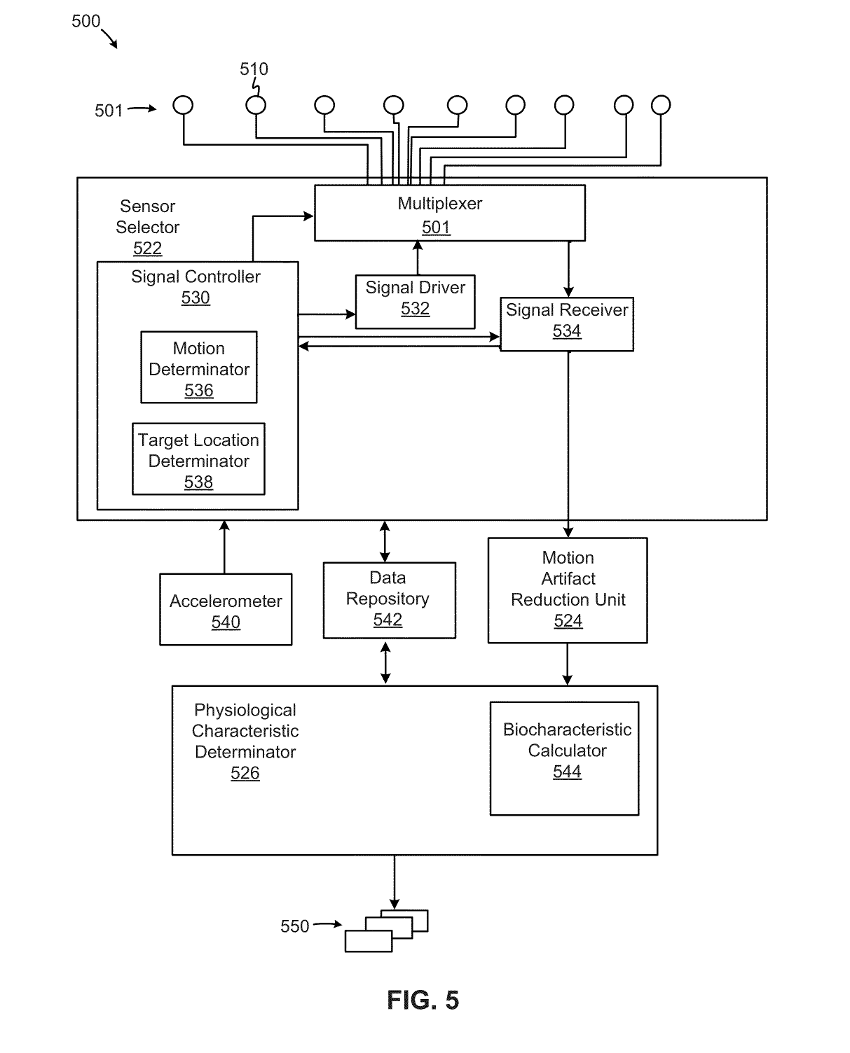

FIG. 5 depicts an example of a physiological information generator, according to some embodiments. Diagram 500 depicts an array 501 of electrodes 510 that can be disposed in a wearable device. A physiological information generator can include one or more of a sensor selector 522, an accelerometer 540 for generating motion data, a motion artifact reduction unit 524, and a physiological characteristic determinator 526. Sensor selector 522 includes a signal controller 530, a multiplexer 501 (or equivalent switching mechanism), a signal driver 532, a signal receiver 534, a motion determinator 536, and a target location determinator 538. Sensor selector 522 is configured to operate in at least two modes. First, sensor selector 522 can select a subset of electrodes in a sensor select mode of operation. Second, sensor selector 522 can use a selected subset of electrodes to acquire physiological characteristics, such as in a data capture mode of operation, according to some embodiments. In sensor select mode, signal controller 530 is configured to serially (or in parallel) configure subsets of electrodes as driver electrodes and sink electrodes, and to cause multiplexer 501 to select subsets of electrodes 510. In this mode, signal driver 532 applies a drive signal via multiplexer 501 to a selected subset of electrodes, from which signal receiver 534 receives via multiplexer 501a sensor signal. Signal controller 530 acquires a data sample for the subset under selection, and then selects another subset of electrodes 510. Signal controller 530 repeats the capture of data samples, and is configured to determine an optimal subset of electrodes for monitoring purposes. Then, sensor selector 522 can operate in the data capture mode of operation in which sensor selector 522 continuously (or substantially continuously) captures sensor signal data from at least one selected subset of electrodes 501 to identify physiological characteristics in real time (or in near real-time).

In some embodiments, a target location determinator 538 is configured to initiate the above-described sensor selection mode to determine a subset of electrodes 510 adjacent a target location. Further, target location determinator 538 can also track displacements of a wearable device in which array 501 resides based on motion data from accelerometer 540. For example, target location determinator 538 can be configured to determine an optimal subset if the initially-selected electrodes are displaced farther away from the target location. In sensor selecting mode, target location determinator 538 can be configured to select another subset, if necessary, by beginning the capture of data samples at electrodes for the last known subset adjacent to the target location, and progressing to other nearby subsets to either confirm the initial selection of electrodes or to select another subset. In some examples, orientation of the wearable device, based on accelerometer data (e.g., a direction of gravity), also can be used to select a subset of electrodes 501 for evaluation as an optimal subset. Motion determinator 536 is configured to detect whether there is an amount of motion associated with a displacement of the wearable device. As such, motion determinator 536 can detect motion and generate a signal to indicate that the wearable device has been displaced, after which signal controller 530 can determine the selection of a new subset that is more closely situated near a blood vessel than other subsets, for example. Also, motion determinator 536 can cause signal controller 530 to disable data capturing during periods of extreme motion (e.g., during which relatively large amounts of motion artifacts may be present) and to enable data capturing during moments when there is less than an extreme amount of motion (e.g., when a tennis player pauses before serving). Data repository 542 can include data representing the gender of the wearer, which is accessible by signal controller 530 in determining the electrodes in a subset.

In some embodiments, signal driver 532 may be a constant current source including an operational amplifier configured as an amplifier to generate, for example, 100 μA of alternating current (“AC”) at various frequencies, such as 50 kHz. Note that signal driver 532 can deliver any magnitude of AC at any frequency or combinations of frequencies (e.g., a signal composed of multiple frequencies). For example, signal driver 532 can generate magnitudes (or amplitudes), such as between 50 μA and 200 μA, as an example. Also, signal driver 532 can generate AC signals at frequencies from below 10 kHz to 550 kHz, or greater. According to some embodiments, multiple frequencies may be used as drive signals either individually or combined into a signal composed of the multiple frequencies. In some embodiments, signal receiver 534 may include a differential amplifier and a gain amplifier, both of which can include operational amplifiers.

Motion artifact reduction unit 524 is configured to subtract motion artifacts from a raw sensor signal received into signal receiver 534 to yield the physiological-related signal components for input into physiological characteristic determinator 526. Physiological characteristic determinator 526 can include one or more filters to extract one or more physiological signals from the raw physiological signal that is output from motion artifact reduction unit 524. A first filter can be configured for filtering frequencies for example, between 0.8 Hz and 3 Hz to extract an HR signal, and a second filter can be configured for filtering frequencies between 0 Hz and 0.5 Hz to extract a respiration signal from the physiological-related signal component. Physiological characteristic determinator 526 includes a biocharacteristic calculator that is configured to calculate physiological characteristics 550, such as VO2 max, based on extracted signals from array 501.

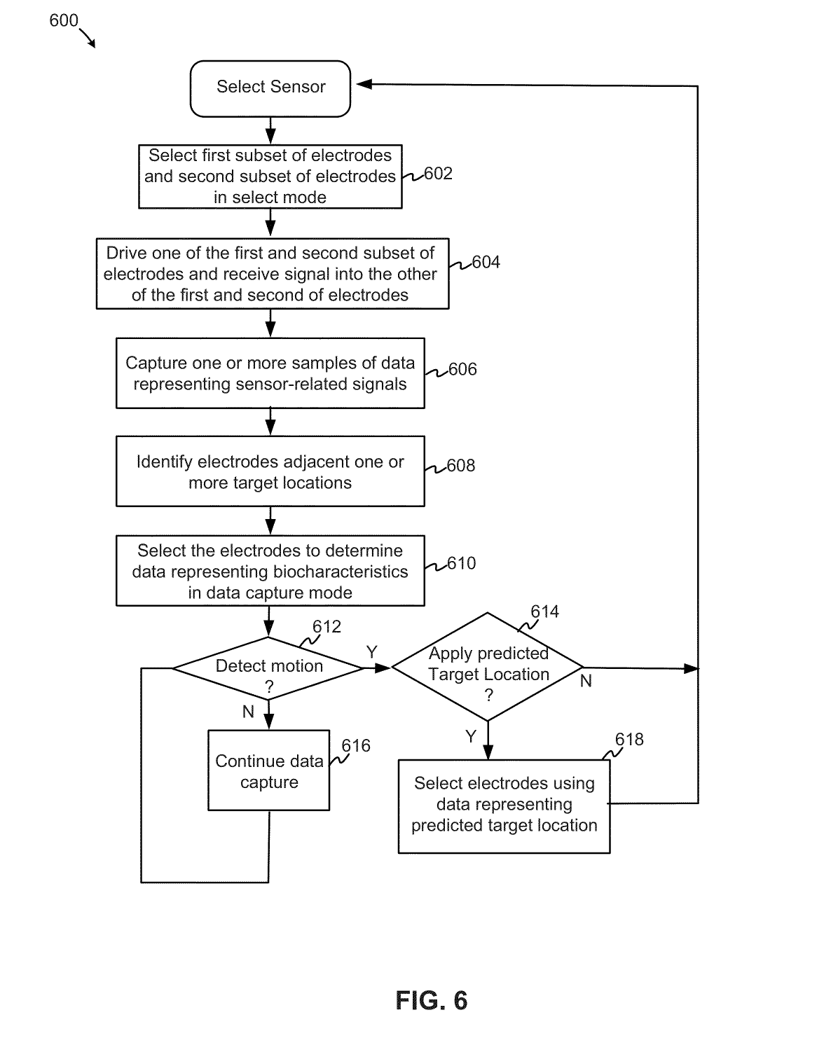

FIG. 6 is an example flow diagram for selecting a sensor, according to some embodiments. At 602, flow 600 provides for the selection of a first subset of electrodes and the selection of a second subset of electrodes in a select sensor mode. At 604, one of the first and second subset of electrodes is selected as a drive electrode and the other of the first and second subset of electrodes is selected as a sink electrode. In particular, the first subset of electrodes can, for example, include one or more drive electrodes, and the second subset of electrodes can include one or more sink electrodes. At 606, one or more data samples are captured, the data samples representing portions of a measured signal (or values thereof). Based on a determination that one of the data samples is indicative of a subset of electrodes adjacent a target location, the electrodes of the optimal subset are identified at 608. At 610, the identified electrodes are selected to capture signals including physiological-relate components. While there is no detected motion at 612, flow 600 moves to 616 to capture, for example, heart and respiration data continuously. When motion is detected at 612, data capture may continue. But flow 600 moves to 614 to determine whether to apply a predicted target location. In some cases, a predicted target location is based on the initial target location (e.g., relative to the initially-determined subset of electrodes), with subsequent calculations based on amounts and directions of displacement, based on accelerometer data, to predict a new target location. One or more motion sensors can be used to determine the orientation of a wearable device, and relative movement of the same (e.g., over a period of time or between events), to determine or predict a target location. Or, the predicted target location can refer to the last known target location and/or subset of electrodes. At 618, electrodes are selected based on the predicted target location for confirming whether the previously-selected subset of electrodes are optimal, or whether a new, optimal subset is to be determined as flow 600 moves back to 602.

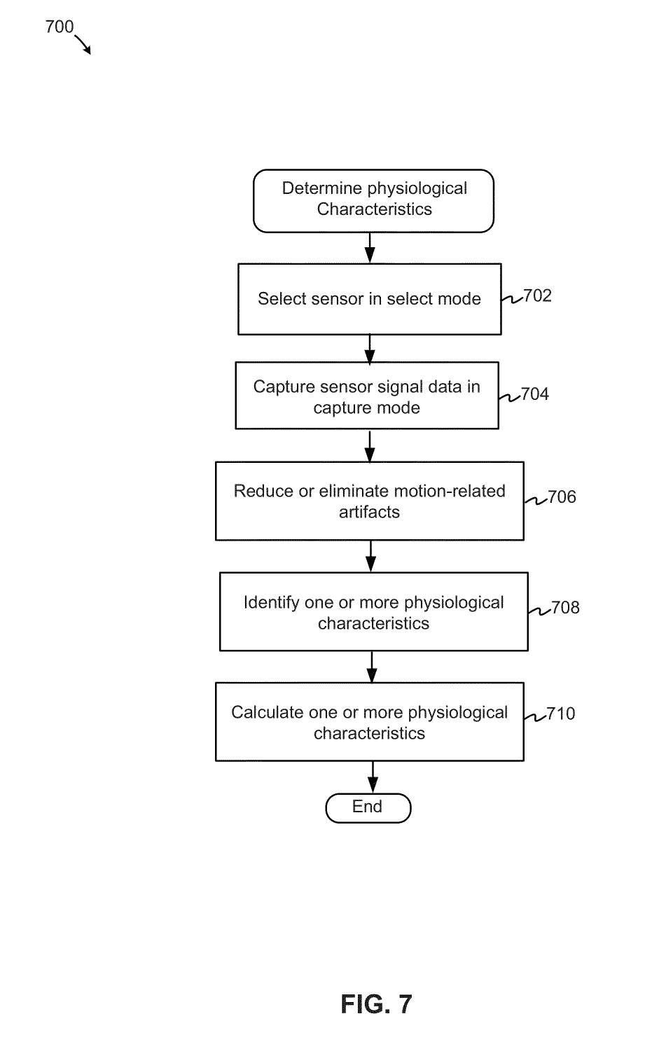

FIG. 7 is an example flow diagram for determining physiological characteristics using a wearable device with arrayed electrodes, according to some embodiments. At 702, flow 700 provides for the selection of a sensor in sensor select mode, the sensor including, for example, two or more electrodes. At 704, sensor signal data is captured in data capture mode. At 706, motion-related artifacts can be reduced or eliminated from the sensor signal to yield a physiological-related signal component. One or more physiological characteristics can be identified at 708, for example, after digitally processing the physiological-related signal component. At 710, one or more physiological characteristics can be calculated based on the data signals extracted at 708. Examples of calculated physiological characteristics include maximal oxygen consumption (“VO2 max”).

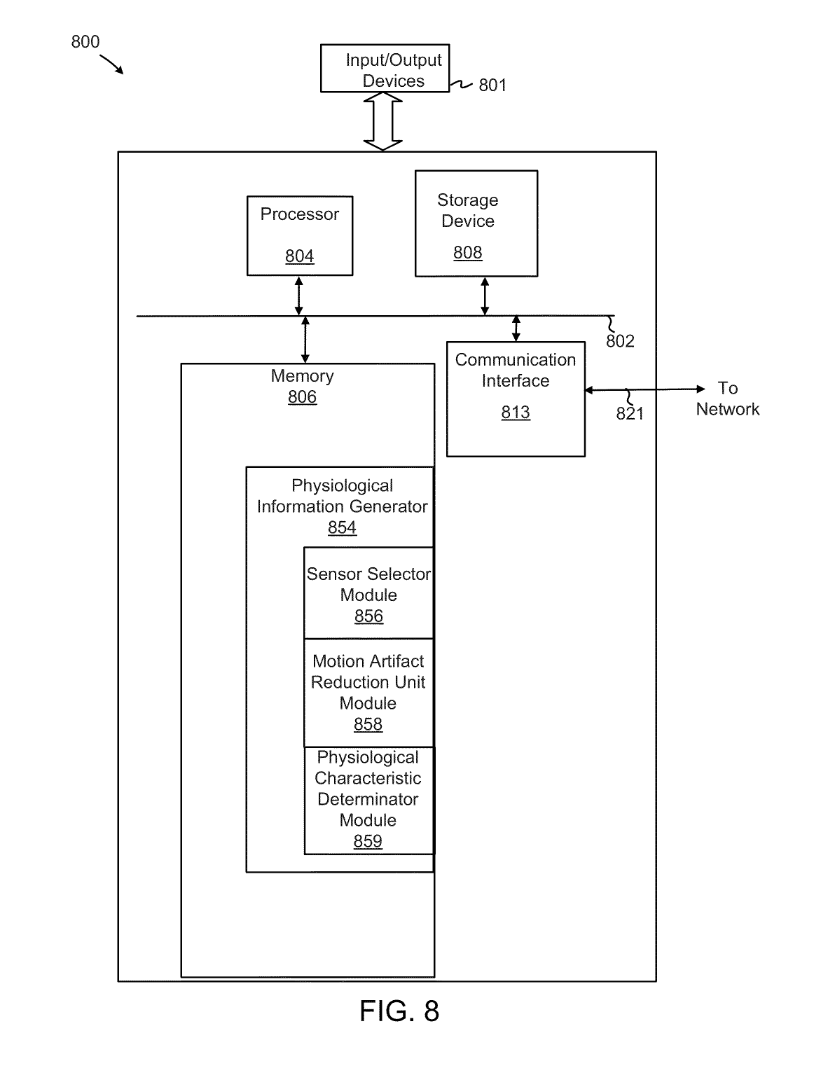

FIG. 8 illustrates an exemplary computing platform disposed in a wearable device in accordance with various embodiments. In some examples, computing platform 800 may be used to implement computer programs, applications, methods, processes, algorithms, or other software to perform the above-described techniques. Computing platform 800 includes a bus 802 or other communication mechanism for communicating information, which interconnects subsystems and devices, such as processor 804, system memory 806 (e.g., RAM, etc.), storage device 808 (e.g., ROM, etc.), a communication interface 813 (e.g., an Ethernet or wireless controller, a Bluetooth controller, etc.) to facilitate communications via a port on communication link 821 to communicate, for example, with a computing device, including mobile computing and/or communication devices with processors. Processor 804 can be implemented with one or more central processing units (“CPUs”), such as those manufactured by Intel® Corporation, or one or more virtual processors, as well as any combination of CPUs and virtual processors. Computing platform 800 exchanges data representing inputs and outputs via input-and-output devices 801, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 800 performs specific operations by processor 804 executing one or more sequences of one or more instructions stored in system memory 806, and computing platform 800 can be implemented in a client-server arrangement, peer-to-peer arrangement, or as any mobile computing device, including smart phones and the like. Such instructions or data may be read into system memory 806 from another computer readable medium, such as storage device 808. In some examples, hard-wired circuitry may be used in place of or in combination with software instructions for implementation. Instructions may be embedded in software or firmware. The term “computer readable medium” refers to any tangible medium that participates in providing instructions to processor 804 for execution. Such a medium may take many forms, including but not limited to, non-volatile media and volatile media. Non-volatile media includes, for example, optical or magnetic disks and the like. Volatile media includes dynamic memory, such as system memory 806.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 802 for transmitting a computer data signal.

In some examples, execution of the sequences of instructions may be performed by computing platform 800. According to some examples, computing platform 800 can be coupled by communication link 821 (e.g., a wired network, such as LAN, PSTN, or any wireless network) to any other processor to perform the sequence of instructions in coordination with (or asynchronous to) one another. Computing platform 800 may transmit and receive messages, data, and instructions, including program code (e.g., application code) through communication link 821 and communication interface 813. Received program code may be executed by processor 804 as it is received, and/or stored in memory 806 or other non-volatile storage for later execution.

In the example shown, system memory 806 can include various modules that include executable instructions to implement functionalities described herein. In the example shown, system memory 806 includes a physiological information generator module 854 configured to implement determine physiological information relating to a user that is wearing a wearable device. Physiological information generator module 854 can include a sensor selector module 856, a motion artifact reduction unit module 858, and a physiological characteristic determinator 859, any of which can be configured to provide one or more functions described herein.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. According to some embodiments, the term “module” can refer, for example, to an algorithm or a portion thereof, and/or logic implemented in either hardware circuitry or software, or a combination thereof. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method comprising:
selecting a subset of electrodes from a plurality of subsets of electrodes, the plurality of subsets of electrodes being disposed in a wearable device;
capturing a sensor signal including data representing a motion-related signal component and a physiological-related component;
generating a motion-related signal at a motion sensor in the wearable device;
reducing a magnitude of the motion-related signal component by an amount associated with the motion-related signal to yield the physiological-related signal component; and
deriving a physiological characteristic from the physiological-related signal component.

2. The method of claim 1, wherein selecting the subset of electrodes comprises:
determining a first subset of electrodes is adjacent to a target location; and
selecting the first subset of electrodes as the subset of electrodes.

3. The method of claim 2, furthering comprising:
determining a second subset of electrodes is adjacent to the target location; and
selecting the second subset of electrodes as the subset of electrodes.

4. The method of claim 3, furthering comprising:
detecting motion of the wearable device.

5. The method of claim 2, wherein determining the first subset of electrodes is adjacent to the target location comprises:
capturing a plurality of data samples each representing a portion of a physiological characteristic;
comparing each of the plurality of data samples to data representing a profile of physiological characteristics;
determining that a data sample captured in association with the first subset of electrodes matches the profile of physiological characteristics relative to other subsets of electrodes; and
identifying the first subset of electrodes being adjacent to the target location.

6. The method of claim 2, wherein determining the first subset of electrodes is adjacent to the target location comprises:
tracking motion of the first subset of electrodes using an accelerometer as the motion sensor relative to a reference;
predicting that the second subset of electrodes is adjacent to the target location;
determining that a data sample captured in association with the second subset of electrodes matches the profile of physiological characteristics relative to other subsets of electrodes; and
identifying the second subset of electrodes being adjacent to the target location.

7. The method of claim 1, wherein deriving the physiological characteristic from the physiological-related signal component comprises:
determining a heart rate (“HR”) signal or a respiration signal as the physiological characteristic.

8. The method of claim 7, wherein deriving the physiological characteristic from the physiological-related signal component further comprises:
calculating a first value representing a maximal oxygen consumption (“VO2 max”) or a second value representing pulse or blood pressure based one or more of the HR and the respiration signal.

9. The method of claim 1, wherein capturing the sensor signal further comprises:
driving a signal through a material of which the wearable device is composed,
wherein the plurality of subsets of electrodes are encapsulated in the material.

10. The method of claim 1, wherein selecting the subset of electrodes further comprises:
selecting electrodes in the subset of the electrodes based on data representing a gender associated with the physiological-related signal component,
wherein the subset of the electrodes are adjacent to a target location adjacent.

11. An apparatus comprising:
a wearable housing;
an array of electrodes disposed substantially in the wearable housing, at least a portion of the array including electrodes configured to either drive a first signal to a target location or receive a second signal from the target location, the second signal including data representing one or more physiological characteristics;
a sensor selector configured to identify a subset of the electrodes adjacent to the target location and to select the subset of the electrodes from which to receive the second signal; and
a signal controller configured to cause the first signal to drive into a first electrode of the subset of the electrodes and the second signal to be received into a second electrode of the subset of electrodes,
wherein the second signal is a sensor signal including data representing one or more physiological characteristics.

12. The apparatus of claim 11, wherein the signal controller is configured further to identify another subset of the electrodes adjacent to the target location and to select the another subset of the electrodes from which to receive the second signal.

13. The apparatus of claim 12, wherein the selection of the another subset of the electrodes indicates a displacement of the subset of the electrodes relative to the target location.

14. The apparatus of claim 13, further comprising:
a motion sensor configured to generate a motion signals; and
a target location determinator configured to track motion of the target location relative to a reference to form an aggregated motion of the target location, and further configured to generate data representing a predicted target location based on the aggregated motion,
wherein sensor selector is configured to use the predicted target location to select another subset of the electrodes as the subset of the electrodes adjacent to the target location.

15. The apparatus of claim 11, further comprising:
a motion sensor configured to generate a motion artifact signal; and
a motion artifact reducer configured to receive the sensor signal including a motion-related component and physiological-related component, and further configured to subtract the motion artifact signal from the sensor signal to remove the motion-related component to obtain the physiological-related component.

16. The apparatus of claim 11, further comprising:
a physiological characteristic determinator configured to derive one or more physiological signals from the sensor signal.

17. The apparatus of claim 16, wherein the physiological characteristic determinator is configured to determine a data signal representing either a heart rate (“HR”) or a respiration signal, or both, as the one or more physiological signals.

18. The apparatus of claim 16, wherein the physiological characteristic determinator is configured to calculate from the sensor signal a first value representing a maximal oxygen consumption (“VO2 max”) or a second value representing pulse or blood pressure.

19. The apparatus of claim 11, wherein the electrodes are configured to capacitively-couple the subset of the electrodes to the target location.

20. The apparatus of claim 11, wherein the wearable housing further comprises:
a material disposed between the electrodes and a surface of the wearable housing configured to confront the target location.
